(19) United States
(12) Patent Application Publication
(10) Pub. No.: US 2023/0212620 A1
(43) Pub. Date: Jul. 6, 2023

(54) Stenotrophomonas pavanii capable of degrading polyethylene terephthalate

(71) Applicant: Jiangnan University, Wuxi (CN)

(72) Inventors: Jing WU, Wuxi (CN); Zhengfei Yan, Wuxi (CN); Qingsong Huang, Wuxi (CN); Lengtao Gu, Wuxi (CN)

(21) Appl. No.: 18/052,599

(22) Filed: Nov. 4, 2022

Related U.S. Application Data
(63) Continuation of application No. PCT/CN2021/097924, filed on Jun. 2, 2021.

(30) Foreign Application Priority Data
Jun. 4, 2020 (CN) 2020104986015

(51) Int. Cl. C12P 7/62; C12N 1/20; C08J 11/10; C12P 7/44

(52) U.S. Cl. CPC C12P 7/62; C12N 1/205; C08J 11/10; C12P 7/44; C12R 2001/01

(57) ABSTRACT

The present disclosure discloses Stenotrophomonas pavanii capable of degrading polyethylene terephthalate, belonging to the technical field of microorganisms. The present disclosure provides a S. pavanii strain JWG-G1 capable of degrading polyethylene terephthalate (PET). After a seed solution of the S. pavanii is inoculated into an inorganic salt liquid medium containing 2 g/L polyethylene terephthalate at an inoculum size of 10% (v/v) and cultured for 5 d, polyethylene terephthalate (PET) particles can be partially degraded into monohydroxyethyl terephthalate and terephthalic acid that can be directly recycled. In addition, ester bond functional groups on the surface of the polyethylene terephthalate plastic particles can be reduced, and a weight loss rate of the polyethylene terephthalate plastic particles can reach 9.4%. Therefore, the S. pavanii JWG-G1 of the present disclosure has very high application prospects in the degradation of polyethylene terephthalate.

The instant application contains a Sequence Listing in XML format as a file named “YGHY-2022-33-SEQ.xml”, created on Aug. 31, 2022, of 4 kB in size, and which is hereby incorporated by reference in its entirety.

### TECHNICAL FIELD

The present disclosure relates to Stenotrophomonas pavanii capable of degrading polyethylene terephthalate, belonging to the technical field of microorganisms.

### BACKGROUND

With the rapid development of economy, people's consumption of plastic products has increased significantly. The annual consumption of plastics in the world exceeds 320 million tons, and the consumption is increasing at an annual rate of 4-6%. Because the plastics are nondegradable, the annual recycling rate of the plastic products in the world is only 14%, which makes plastic waste accumulate continuously in the environment and poses a serious threat to the ecology.

Polyethylene terephthalate (PET) is a linear macromolecule formed by sequentially linking ethylene glycol (EG) and terephthalic acid (TPA) by ester bonds. At present, polyethylene terephthalate (PET) plastic products account for about 60% of all plastic products. Correspondingly, polyethylene terephthalate (PET) plastic waste also accounts for a relatively high proportion of all plastic waste. Therefore, the degradation of polyethylene terephthalate (PET) is crucial to the governance of plastic waste.

Biodegradation technology is a technology of directly degrading plastics by strains capable of degrading plastics. Because of its advantages of no pollution and low cost, it has gradually become a research hotspot in the field of degradation of plastics. These strains include, for example, Ideonella sakaiensis strain 201-F6, capable of using polyethylene terephthalate (PET) as the sole nutrient source and having a degradation rate of 0.13 mg/d (Science, 2016, 351(6278): 1196-1199); Ochrobactrum strains capable of degrading polylactic acid (PLA) (publication number: CN102639690A); and Penicillium strains capable of degrading polyhydroxyalkanoate (PHA) (Polymer-plastics Technology and Engineering, 2009, 48 (1): 58-63).

However, compared with bio-based plastics such as polyhydroxyalkanoate (PHA) and polylactic acid (PLA), which are also formed by linking with C—O bonds, the molecular chain of polyethylene terephthalate (PET) contains a large number of aromatic groups, which makes the steric hindrance of the molecular chain high and the surface more hydrophobic. Therefore, it is difficult to degrade PET by microorganisms. As a result, it is still difficult to obtain strains capable of degrading polyethylene terephthalate (PET).

### SUMMARY

The present disclosure provides a S. pavanii strain JWG-G1, which has been deposited in China Center for Type Culture Collection on Jun. 3, 2019 with the deposit number of CCTCC NO: M 2019415.

The S. pavanii JWG-G1 is isolated from a soil sample from Taohuashan Landfill in Wuxi. After sequencing analysis, the 16S rDNA sequence of the strain is shown in SEQ ID NO.1. The sequence obtained by sequencing is aligned for nucleic acid sequences in Genbank, and the result shows that the similarity with the nucleic acid sequence of Stenotrophomonas is up to 99%. A phylogenetic tree is constructed using strains having high similarities with the Stenotrophomonas (as shown in FIG. 1), and the result shows that the strain belongs to S. pavanii in the genus Stenotrophomonas, and is named Stenotrophomonas pavanii JWG-G1.

The colony of the S. pavanii JWG-G1 on an LB solid medium is round and convex, pale yellow, opaque, moist and shiny, with flagella on the edge (as shown in FIG. 2-FIG. 3).

The present disclosure further provides application of the above S. pavanii JWG-G1 in degrading polyethylene terephthalate, gelatin or esculin.

In an embodiment of the present disclosure, the present disclosure further provides a method for degrading polyethylene terephthalate (PET). The method includes: inoculating a seed solution of the above S. pavanii JWG-G1 into a liquid medium containing the polyethylene terephthalate, and carrying out culturing.

In an embodiment of the present disclosure, the seed solution of the above S. pavanii JWG-G1 is inoculated in the liquid medium containing the polyethylene terephthalate at an inoculum size of not less than 10% (v/v).

In an embodiment of the present disclosure, a concentration of the seed solution of the above S. pavanii JWG-G1 in the medium is not less than 1×10⁸ CFU/mL.

In an embodiment of the present disclosure, the medium is an inorganic salt medium.

In an embodiment of the present disclosure, the present disclosure further provides a product applicable to degrading polyethylene terephthalate (PET). The product contains the above S. pavanii JWG-G1.

The present disclosure further provides a method for hydrolyzing gelatin. The method includes: inoculating the above S. pavanii JWG-G1 into a plate medium containing gelatin, and carrying out culturing.

The present disclosure further provides a product applicable to hydrolyzing gelatin. The product contains the above S. pavanii JWG-G1.

The present disclosure further provides a method for hydrolyzing esculin. The method includes: inoculating the above S. pavanii JWG-G1 into a plate medium containing esculin, and carrying out culturing.

The present disclosure further provides a product applicable to hydrolyzing esculin. The product contains the above S. pavanii JWG-G1.

### Beneficial Effects

(1) The present disclosure provides a S. pavanii strain JWG-G1 capable of degrading polyethylene terephthalate (PET). After the seed solution of the S. pavanii JWG-G1 is inoculated into the inorganic salt liquid medium containing 2 g/L polyethylene terephthalate (PET) at an inoculum size of 10% (v/v) and cultured for 5 d, polyethylene terephthalate (PET) particles can be partially degraded into monohydroxyethyl terephthalate (MHET) and terephthalic acid (TPA) that can be directly recycled. In addition, under the action of the S. pavanii JWG-G1 of the present disclosure, ester bond functional groups on the surface of the polyethylene terephthalate (PET) plastic particles can be reduced, and a weight loss rate of the polyethylene terephthalate (PET) plastic particles can reach 9.4%. Therefore, the S. pavanii JWG-G1 of the present disclosure has very high application prospects in the degradation of polyethylene terephthalate (PET).

(2) The S. pavanii JWG-G1 of the present disclosure has excellent salt tolerance and can grow vigorously in an LB liquid medium containing 1-9 g/L NaCl.

(3) The S. pavanii JWG-G1 of the present disclosure is capable of hydrolyzing gelatin and esculin.

Biological Material Deposit

A S. pavanii strain JWG-G1, taxonomically named Stenotrophomonas pavanii JWG-G1, has been deposited in China Center for Type Culture Collection on Jun. 3, 2019 with the deposit number of CCTCC NO: M 2019415, at Wuhan University, Wuhan, China.

### BRIEF DESCRIPTION OF FIGURES

FIG. 1 shows a phylogenetic tree of S. pavanii JWG-G1;

FIG. 2 shows colonies of the S. pavanii JWG-G1;

FIG. 3 shows cell morphology of the S. pavanii JWG-G1;

FIG. 4 shows growth curves of the S. pavanii JWG-G1 at different pH values;

FIG. 5 shows growth curves of the S. pavanii JWG-G1 at different temperatures;

FIG. 6 shows changes of ester bond functional groups after the surface of polyethylene terephthalate (PET) plastic particles is treated with the S. pavanii JWG-G1;

FIG. 7 shows hydrolysis rate of gelatin by the S. pavanii JWG-G1JWG-G1; and

FIG. 8 shows hydrolysis rate of esculin by the S. pavanii JWG-G1JWG-G1.

### DETAILED DESCRIPTION

The present disclosure will be further described below in conjunction with specific examples.

Diethyl terephthalate (DET), polyethylene terephthalate (PET) plastic particles and bis-hydroxyethyl terephthalate (BHET) involved in the following examples were purchased from Sigma, and standards of TPA, MHET and BHET were purchased from Sigma.

Media involved in the following examples are as follows:

LB solid medium (g/L): peptone 10, yeast powder 5, sodium chloride 10 and agar 13, pH 7.0.

LB liquid medium (g/L): peptone 10, yeast powder 5 and sodium chloride 10, pH 7.0.

Inorganic salt liquid medium (g/L): KH₂PO₄ 0.7, K₂HPO₄·3H₂O 0.5, NH₄Cl 2, MgSO₄·7H₂O 0.6, NaCl 0.005, FeSO₄·7H₂O 0.001, ZnSO₄.7H₂O 0.002 and MnSO₄·H₂O 0.001.

Inorganic salt solid medium (g/L): KH₂PO₄ 0.7, K₂HPO₄·3H₂O 0.5, NH₄Cl 2, MgSO₄·7H₂O 0.6, NaCl 0.005, FeSO₄·7H₂O 0.001, ZnSO₄·7H₂O 0.002, MnSO₄·H₂O 0.001 and agar powder 13.

Test Methods Involved in the Following Examples are as Follows:

Test Method of Changes of Functional Groups on Surface of Polyethylene Terephthalate (PET) Plastic Particles:

The polyethylene terephthalate (PET) plastic particles treated by the strain were repeatedly washed with deionized water 3-4 times. After being washed, the polyethylene terephthalate (PET) plastic particles were ultrasonicated at a power of 200 W and a frequency of 58 KHz for 15 min. After being ultrasonicated, the polyethylene terephthalate (PET) plastic particles were dried in an oven at 60° C. for 6 h. Untreated polyethylene terephthalate (PET) plastic particles were taken as a control. The surface of the untreated polyethylene terephthalate (PET) plastic particles and the surface of the polyethylene terephthalate (PET) plastic particles treated by the strain were tested for the changes of functional groups using a Fourier transform infrared spectrometer.

Test Method of Degradation Products and Contents Thereof:

Treatment of standards: The standards of TPA, MHET and BHET were respectively weighed and dissolved in dimethyl sulfoxide (DMSO) to obtain a mother solution. The mother solution was diluted with sterile water to obtain a 0.1 mg/mL standard solution. The standard solution was filtered with a 0.22 μM filter. The filtrate was injected into a liquid phase bottle with a syringe for HPLC detection.

Treatment of samples: The culture solution was allowed to stand for 10 min. 5 mL of supernatant was taken and centrifuged at 12000 rpm for 8 min. Filtration with a 0.22 μM filter was carried out. The filtrate was injected into a liquid phase bottle with a syringe for HPLC detection.

Test Method of Weight Loss Rate:

Weight loss rate of PET plastic particles (%)=[(m2−m1)÷m2]×100.

m1: The PET plastic particles treated by the strain were repeatedly washed with deionized water 3-4 times. After being washed, the PET plastic particles were ultrasonicated at a power of 200 W and a frequency of 58 KHz for 15 min, dried in an oven at 60° C. for 6 h and then weighed.

m2: The untreated PET plastic particles were repeatedly washed with deionized water 3-4 times. After being washed, the PET plastic particles were ultrasonicated at a power of 200 W and a frequency of 58 KHz for 15 min, dried in an oven at 60° C. for 6 h and then weighed.

Hydrolysis Rates of Gelatin and Esculin:

Hydrolysis rate (cm/day)=D1/n

D1: on the nth day, the diameter of the clear zone on the plate (cm); n: the number of days of plate culture (day).

### Example 1: Screening of S. pavanii

Specific steps were as follows:

1. General Screening

Soil from Taohuashan Landfill in Wuxi was taken as a sample. 1 g of the soil was added to 9 mL of normal saline, and subjected to enrichment culture at 35° C. and 180 rpm for 30 min while shaking. Then, 1 mL of supernatant was taken and sequentially diluted to 10⁻⁴, 10⁻⁵ and 10⁻⁶. 200 μL of dilute solution that had been diluted to 10⁻⁴, 10⁻⁵ and 10⁻⁶ was respectively taken, uniformly spread on an inorganic salt solid medium containing 2 g/L polyethylene terephthalate (PET), and cultured at a constant temperature in a 35° C. incubator until colonies grew out. The experimental result showed that after days of culture, no colonies appeared on the plate for isolation, and this method could not effectively isolate the strains with the ability to degrade polyethylene terephthalate (PET).

2. “PET-Induced Culture” Screening

(1) Soil from Taohuashan Landfill in Wuxi was used as a sample. 1 g of the soil was added to 9 mL of inorganic salt liquid medium containing 2 g/L polyethylene terephthalate (PET), and subjected to enrichment culture at 35° C. and 180 rpm for 48 h while shaking. Next, 1 mL of enrichment solution was pipetted and added to 9 mL of fresh inorganic salt liquid medium containing 2 g/L polyethylene terephthalate (PET), and culture was carried out for 10 cycles under the same conditions.

(2) Then, 1 mL of supernatant of the culture solution obtained after 10 cycles of culture in step (1) was taken and sequentially diluted to 10⁻⁴, 10⁻⁵ and 10⁻⁶. 200 μL of dilute solution that had been diluted to 10⁻⁴, 10⁻⁵ and 10⁻⁶ was respectively taken, uniformly spread on an inorganic salt solid medium containing 2 g/L polyethylene terephthalate (PET), and cultured at a constant temperature in a 35° C. incubator until colonies grew out. The experimental result showed that after days of culture, there were sparse colonies in the plate medium for isolation (5-6 colonies/plate).

(3) As a control, 1 mL of supernatant of the culture solution obtained after 10 cycles of culture in step (1) was taken and sequentially diluted to 10⁻⁴, 10⁻⁵ and 10⁻⁶. 200 μL of dilute solution that had been diluted to 10⁻⁴, 10⁻⁵ and 10⁻⁶ was respectively taken, transferred and inoculated into an inorganic salt solid medium without polyethylene terephthalate (PET), and cultured at 35° C. All the colonies above could grow on the inorganic salt solid medium without polyethylene terephthalate (PET).

As can be seen, all the colonies above were autotrophic purified strains, rather than non-autotrophic strains using PET as the sole nutrient source. This method could not effectively isolate the strains with the ability to degrade polyethylene terephthalate (PET).

3. “Level-by-Level Screening” Strategy

(1) Soil from Taohuashan Landfill in Wuxi was used as a sample. 1 g of the soil was added to 9 mL of inorganic salt liquid medium containing 10 g/L diethyl terephthalate (DET), and subjected to enrichment culture at 35° C. and 180 rpm for 48 h while shaking. Next, 1 mL of enrichment solution was pipetted and added to 9 mL of fresh inorganic salt liquid medium containing 10 g/L diethyl terephthalate (DET), and culture was carried out for 10 cycles under the same conditions.

(2) 1 mL of supernatant of the culture solution obtained after 10 cycles of culture in step (1) was pipetted and added to 9 mL of fresh inorganic salt liquid medium containing bis-hydroxyethyl terephthalate (BHET), and cultured for 10 cycles under the same conditions.

(3) 1 mL of supernatant of the culture solution obtained after 10 cycles of culture in step (2) was pipetted and added to 9 mL of fresh inorganic salt liquid medium containing 2 g/L polyethylene terephthalate (PET), and cultured for 10 cycles under the same conditions.

(4) 1 mL of supernatant of the culture solution obtained after 10 cycles of culture in step (3) was taken and sequentially diluted to 10⁻⁴, 10⁻⁵ and 10⁻⁶. 200 μL of dilute solution that had been diluted to 10⁻⁴, 10⁻⁵ and 10⁻⁶ was respectively taken, uniformly spread on an inorganic salt solid medium containing 2 g/L polyethylene terephthalate (PET), and cultured at a constant temperature in a 35° C. incubator until colonies grew out (180-200 colonies/plate). Using an inorganic salt solid medium without any nutrient source as a control, 10 non-autotrophic purified strains were obtained, and the purified strain with the best growth vigor was respectively named JWG-G1.

Based on the results of the 3 different isolation and screening method above, it could be found that it was required to use the soluble and simple-structured polyethylene terephthalate (PET) intermediates such as diethyl terephthalate (DET) and bis-hydroxyethyl terephthalate (BHET) as the preliminary substrates to enrich the strain capable of degrading polyethylene terephthalate (PET), such that the strain adapted the polyethylene terephthalate (PET) intermediates as the sole nutrient. Then, the strain was enriched again using polyethylene terephthalate (PET) as the sole substrate, so that the strain capable of degrading polyethylene terephthalate (PET) was derived from the strain capable of degrading polyethylene terephthalate (PET) intermediates and its concentration was further increased. Therefore, the “level-by-level screening” strategy could effectively screen out the strain with the ability to degrade PET.

### Example 2: Identification of S. pavanii

Total DNA of the strain JWG-G1 was extracted and subjected to 16S rDNA amplification and sequencing (completed by Wuxi Tianlin Biotechnology Co., Ltd.). The sequence obtained by sequencing was aligned for nucleic acid sequences in Genbank. It was found that the strain JWG-G1 had a 16S rDNA sequence homology of greater than 99% with Stenotrophomonas, and had a 16S rDNA similarity of up to 99.5% to S. pavanii DSM 25135. As a result, the strain JWG-G1 belonged to the genus Stenotrophomonas.

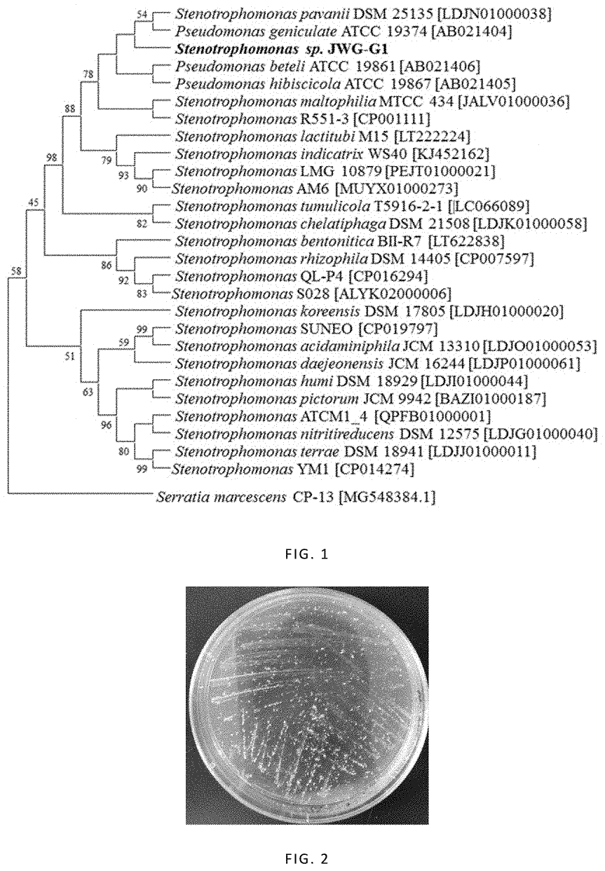

A phylogenetic tree was constructed using strains having high similarities with the 16S rDNA sequence (as shown in SEQ NO.1) of the strain JWG-G1 (the phylogenetic tree constructed based on the strain JWG-G1 was shown in FIG. 1). The result showed that the strain JWG-G1 and the S. pavanii DSM 25135 belonged to the same branch. As a result, the strain JWG-G1 belonged to the genus Stenotrophomonas, and named Stenotrophomonas pavanii JWG-G1.

### Example 3: Culture of S. pavanii

Specific steps were as follows:

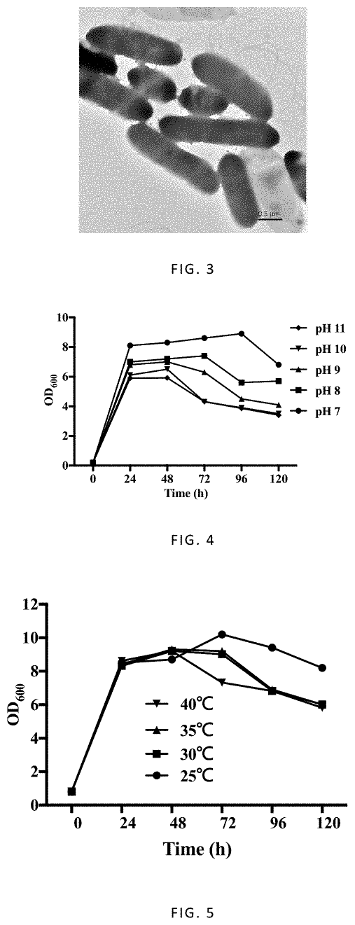

A ring of S. pavanii JWG-G1 obtained in Example 1 was scraped and inoculated into an LB solid medium for streak culture. After 36 h of culture at 35° C., the colonies were observed. It was found that the colony was round and convex, pale yellow, opaque, moist and shiny, with flagella on the edge (as shown in FIG. 2-FIG. 3).

The S. pavanii JWG-G1 obtained in Example 1 was gram-stained and observed under a microscope, and it was found that the strain was a gram-positive bacterium.

A ring of S. pavanii JWG-G1 obtained in Example 1 was scraped and inoculated into LB liquid media respectively having a pH of 7-11. After culture at 35° C., OD₆₀₀ in the culture solution was tested by a microplate reader. It was found that the optimal growth pH was 7 (as shown in FIG. 4).

A ring of S. pavanii JWG-G1 obtained in Example 1 was scraped and inoculated into LB liquid media having a pH of 7. After culture respectively at 25-40° C., OD₆₀₀ in the culture solution was tested by a microplate reader. It was found that the optimum growth temperature was 25° C. (as shown in FIG. 5).

### Example 4: Degradation Abilities of Different Stenotrophomonas and S. pavanii to Polyethylene Terephthalate (PET) Plastic Particles

Specific steps were as follows:

As the S. pavanii JWG-G1 belongs to the genus Stenotrophomonas which may be one of the potential source genera of strains for degrading PET plastic particles, 7 Stenotrophomonas strains (S. pavanii DSM 25135; Stenotrophomonas bentonitica DSM 103927; Stenotrophomonas chelatiphaga DSM 21508; Stenotrophomonas ginsengisoli KCTC 12539; Stenotrophomonas lactitubi DSM 104152; Stenotrophomonas rhizophila DSM 14405; Stenotrophomonas tumulicola NCIMB 15009) having close genetic relationship with the S. pavanii JWG-G1 were collected and used together with the S. pavanii JWG-G1 strain as the test strains.

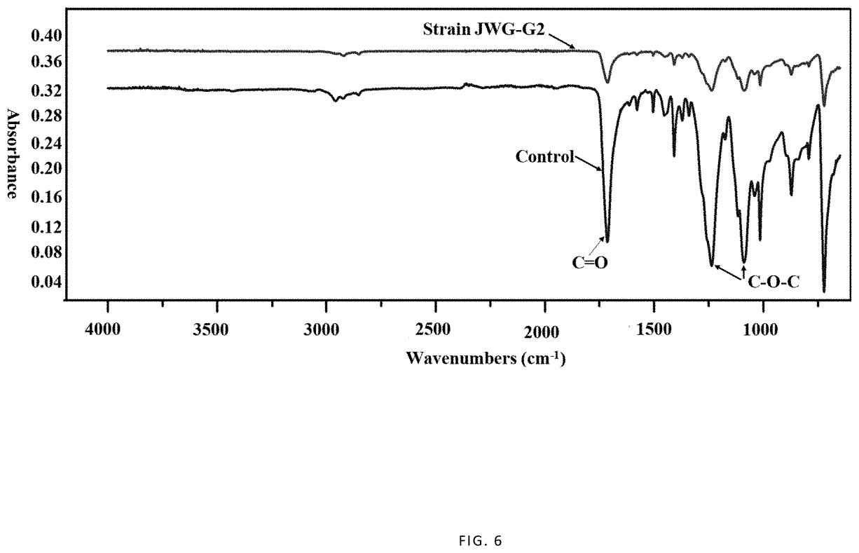

Single colonies of the S. pavanii JWG-G1 and the 7 Stenotrophomonas strains obtained in Example 1 were picked up and respectively inoculated into 100 mL of LB liquid medium, and subjected to shake culture at 35° C. and 180 rpm for 24 h to obtain a seed solution A. The seed solution A was transferred and inoculated into 100 mL of fresh LB liquid medium at an inoculum size of 10% (v/v), and subjected to shake culture at 35° C. and 180 rpm for 24 h to obtain a culture solution A. The culture solution A was centrifuged at 8000 rpm for 10 min, and cells were collected. The cells were washed with an inorganic salt medium 2 times and then made into a bacterial suspension with OD₆₀₀=1.0 as a seed solution B. An inorganic salt liquid medium containing 2 g/L polyethylene terephthalate (PET) without the seed solution B was used as a control group. The seed solution B was inoculated into an inorganic salt liquid medium containing 2 g/L polyethylene terephthalate (PET) at an inoculum size of 10% (v/v) (at this time, the cell concentration of S. pavanii JWG-G1 of the seed solution B in the inorganic salt liquid medium containing PET was 1×10⁹ CFU/mL), and subjected to shake culture at 35° C. and 180 rpm for 5 d to obtain a culture solution B. The changes in OD₆₀₀ of the culture solution B before and after culture were shown in Table 1. The PET plastic particles in the culture solution B were taken out, and tested for the changes in the functional group structure on the surface (the changes in the ester bond functional group structure on the surface of the polyethylene terephthalate (PET) plastic particles in the culture solutions B obtained by culturing the S. pavanii JWG-G1 and the 7 Stenotrophomonas strains were in shown Table 1, and the change in the functional group structure on the surface of the PET plastic particles in the culture solution B obtained by culturing the S. pavanii JWG-G1 was shown in FIG. 6) and their weight loss rate (the weight loss rates of the polyethylene terephthalate (PET) plastic particles in the culture solutions B obtained by culturing the S. pavanii JWG-G1 and the 7 Stenotrophomonas strains were shown in Table 1). Also, the contents of the degradation products of the polyethylene terephthalate (PET) plastic particles, namely monohydroxyethyl terephthalate (MHET) and terephthalic acid (TPA), in the culture solution B were tested (the contents of the degradation products of the polyethylene terephthalate (PET) plastic particles, namely monohydroxyethyl terephthalate (MHET) and terephthalic acid (TPA), in the culture solution B obtained by culturing the S. pavanii JWG-G1 and the 7 Stenotrophomonas strains were shown in Table 1).

As can be seen from Table 1 and FIG. 6, when the PET plastic particles were used as the sole nutrient source, the S. pavanii JWG-G1 could grow; but the OD₆₀₀ of the 7 Stenotrophomonas strains did not change significantly (±0.04 within the error range). As a result, only the S. pavanii JWG-G1 could grow and reproduce using the PET plastic particles as the sole nutrient source.

As can be seen from Table 1 and FIG. 6, after being treated by the S. pavanii JWG-G1 for 5 d, the polyethylene terephthalate (PET) plastic particles were partially degraded into monohydroxyethyl terephthalate (MHET) and terephthalic acid (TPA), the ester bond functional groups on the surface of the polyethylene terephthalate (PET) plastic particles were destroyed (there were two characteristic peaks between 1000 and 1300 cm-1, and one characteristic peak between 1700 and 1750 cm-1), and the weight loss rate of the polyethylene terephthalate (PET) plastic particles was 9.4%; But after being treated by the 7 Stenotrophomonas strains for 5 d, the polyethylene terephthalate (PET) plastic particles did not change significantly. As a result, only the S. pavanii JWG-G1 could degrade the polyethylene terephthalate (PET) plastic particles.

TABLE 1
Performance test before and after culturing S. pavanii JWG-
G1 and 7 Stenotrophomonas strains in media containing PET

| | | | | Ester | |
|---|---|---|---|---|---|
| | | Degradation | | bond | Weight |
| | | product | | func- | loss rate |
| | | (mg/L) | | tional | of PET |
| Strain | OD₆₀₀ | TPA | MHET | group | (%) |
| Stenotrophomonas pavanii | 1.2 | 181 | 7.8 | + | 9.4 |
| JWG-G1 | | | | | |
| Stenotrophomonas pavanii | 0.03 | − | − | − | − |
| DSM 25135 | | | | | |
| Stenotrophomonas | 0.02 | − | − | − | − |
| bentonitica DSM 103927 | | | | | |
| Stenotrophomonas | 0.01 | − | − | − | − |
| chelatiphaga DSM 21508 | | | | | |
| Stenotrophomonas | 0.02 | − | − | − | − |
| ginsengisoli KCTC 12539 | | | | | |
| Stenotrophomonas lactitubi | 0.02 | − | − | − | − |
| DSM 104152 | | | | | |
| Stenotrophomonas | 0.01 | − | − | − | − |
| rhizophila DSM 14405 | | | | | |
| Stenotrophomonas | 0.01 | − | − | − | − |
| tumulicola NCIMB 15009 | | | | | |

The change in OD₆₀₀ before and after culturing the S. pavanii JWG-G1 and the 7 Stenotrophomonas strains in the inorganic salt liquid medium containing 2 g/L polyethylene terephthalate (PET) was the OD₆₀₀ after culturing the S. pavanii JWG-G1 and the 7 Stenotrophomonas strains in the inorganic salt liquid medium containing 2 g/L polyethylene terephthalate (PET) minus the OD₆₀₀ before culturing the S. pavanii JWG-G1 and the 7 Stenotrophomonas strains in the inorganic salt liquid medium containing 2 g/L polyethylene terephthalate (PET). “+”: tested positive; and “−”: tested negative.

### Example 5: Degradation Abilities of S. pavanii JWG-G1 to Different Contents of Polyethylene Terephthalate (PET) Plastic Particles

The specific embodiment was the same as in Example 4, except that the seed solution of the S. pavanii JWG-G1 was respectively inoculated into inorganic salt liquid media containing 2.5 g/L, 3.0 g/L, 3.5 g/L and 4.0 g/L polyethylene terephthalate (PET) at an inoculum size of 10% (v/v).

The results were shown in Table 2. In the degradation products respectively obtained after degrading 2.5 g/L, 3.0 g/L, 3.5 g/L and 4.0 g/L polyethylene terephthalate (PET) by the S. pavanii, the contents of terephthalic acid (TPA) were respectively 203 mg/L, 206 mg/L, 205 mg/L and 205 mg/L, the contents of monohydroxyethyl terephthalate (MHET) were respectively 8.1 mg/L, 8.0 mg/L, 8.3 mg/L and 8.2 mg/L, and the weight loss rates of polyethylene terephthalate (PET) particles respectively reached 9.8%, 9.7%, 9.6% and 9.7%. The results showed that the degradation effect of the S. pavanii JWG-G1 on polyethylene terephthalate (PET) did not increase with the increase of the polyethylene terephthalate content.

TABLE 2
Degradation abilities of S. pavanii to different contents
of polyethylene terephthalate (PET) plastic particles

| Amount of | Polyethylene | Degradation product | | Weight loss |
|---|---|---|---|---|
| S. pavanii | terephthalate | (mg/L) | | rate of PET |
| JWG-G1 (%) | content (g/L) | TPA | MHET | (%) |
| 10 | 2.5 | 203 | 8.1 | 9.8 |
| | 3.0 | 206 | 8.0 | 9.7 |
| | 3.5 | 205 | 8.3 | 9.6 |
| | 4.0 | 205 | 8.2 | 9.7 |

### Example 6: Degradation Abilities of Different Amounts of S. pavanii to Polyethylene Terephthalate (PET) Plastic Particles

The specific embodiment was the same as in Example 4, except that the seed solution of S. pavanii JWG-G1 was inoculated into the inorganic salt liquid medium containing 2 g/L polyethylene terephthalate (PET) at an inoculum size of 15%, 20%, 25% and 30% (v/v).

The results were shown in Table 3. In the degradation products obtained after degrading 2 g/L polyethylene terephthalate (PET) by the S. pavanii respectively at an inoculum size of 15%, 20%, 25% and 30% (v/v), the contents of terephthalic acid (TPA) were respectively 250 mg/L, 300 mg/L, 330 mg/L and 360 mg/L, the contents of monohydroxyethyl terephthalate (MHET) were respectively 8.1 mg/L, 10.6 mg/L, 12.3 mg/L and 15.2 mg/L, and the weight loss rates of polyethylene terephthalate (PET) particles respectively reached 10.3%, 11.6%, 12.3% and 13.9%. The results showed that as the inoculum size of the S. pavanii JWG-G1 increased, the degradation effect of polyethylene terephthalate (PET) became more significant.

TABLE 3
Degradation abilities of different amounts of S. pavanii
to polyethylene terephthalate (PET) plastic particles

| Amount of | Polyethylene | Degradation product | | Weight loss |
|---|---|---|---|---|
| S. pavanii | terephthalate | (mg/L) | | rate of PET |
| JWG-G1 (%) | content (g/L) | TPA | MHET | (%) |
| 15 | 2 | 250 | 8.1 | 10.3 |
| 20 | | 300 | 10.6 | 11.6 |
| 25 | | 330 | 12.3 | 12.3 |
| 30 | | 360 | 15.2 | 13.9 |

### Example 7: Salt Tolerance of S. pavanii

Specific steps were as follows:

A single colony of the S. pavanii JWG-G1 obtained in Example 1 was picked up and inoculated into 100 mL of LB liquid medium, and subjected to shake culture at 35° C. and 180 rpm for 24 h to obtain a seed solution A. The seed solution A was transferred and inoculated into 100 mL of fresh LB liquid medium at an inoculum size of 10% (v/v), and subjected to shake culture at 35° C. and 180 rpm for 72 h to obtain a culture solution A. The culture solution A was centrifuged at 8000 rpm for 10 min, and cells were collected. The cells were washed with an inorganic salt medium 2 times and then made into a bacterial suspension with OD₆₀₀=1.0 as a seed solution B. The seed solution B was respectively inoculated at an inoculum size of 10% (v/v) to LB liquid media containing different concentrations of NaCl (1 g/L, 2 g/L, 3 g/L, 4 g/L, 5 g/L, 6 g/L, 7 g/L, 8 g/L, 9 g/L and 10 g/L), and subjected to shake culture at 35° C. and 180 rpm for 5 d to obtain a culture solution B.

The OD₆₀₀ of the culture solution B was tested. The results showed that the OD₆₀₀ in the culture solution B obtained by culturing the S. pavanii JWG-G1 in the LB liquid media containing 1-9 g/L NaCl for 5 d was increased respectively by 0.20, 10.31, 0.35, 0.45, 0.35, 0.32, 0.31, 0.29, 0.24 and 0.21. As a result, the S. pavanii JWG-G1 has excellent salt tolerance.

### Example 8: Abilities of S. pavanii to Hydrolyze Gelatin and Esculin

Specific steps were as follows:

The abilities of the S. pavanii JWG-G1 to hydrolyze gelatin and esculin were tested by an on-plate clear zone method.

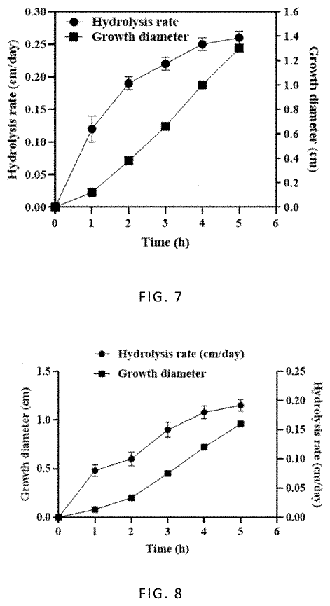

As can be seen from the test results, after the S. pavanii grew on plate media respectively containing gelatin and esculin, an obvious clear zone of hydrolysis appeared around the colony. After 5 d of culture, the diameter of the clear zone of gelatin hydrolysis reached 1.3 cm, and the hydrolysis rate of gelatin by the strain JWG-G1 was 0.26 cm/day (FIG. 7). The diameter of the clear zone of esculin hydrolysis reached 0.9 cm, and the hydrolysis rate of esculin by the strain JWG-G1 was 0.18 cm/day (FIG. 8). As a result, the S. pavanii JWG-G1 had the abilities to hydrolyze gelatin and esculin.

Although the present disclosure has been disclosed as above by way of the preferred examples, they are not intended to limit the present disclosure. Any person skilled in the art can make various changes and modifications without departing from the spirit and scope of the present disclosure. Therefore, the protection scope of the present disclosure should be as defined in the claims.

What is claimed is:

1. Application of Stenotrophomonas pavanii in degrading polyethylene terephthalate, gelatin or esculin, comprising: inoculating S. pavanii into a liquid medium containing the polyethylene terephthalate, gelatin or esculin and carrying out culturing, wherein the S. pavanii has been deposited in China Center for Type Culture Collection on Jun. 3, 2019 with the deposit number of CCTCC NO: M 2019415.

2. A method for degrading polyethylene terephthalate, comprising: inoculating S. pavanii into a liquid medium containing the polyethylene terephthalate and carrying out culturing, wherein the S. pavanii has been deposited in China Center for Type Culture Collection on Jun. 3, 2019 with the deposit number of CCTCC NO: M 2019415.

3. The method according to claim 2, wherein the S. pavanii is inoculated into the liquid medium containing the polyethylene terephthalate in the form of a seed solution; and a volume of the seed solution accounts for not less than 10% of a total volume of the liquid medium.

4. The method according to claim 3, wherein in the seed solution, a concentration of the S. pavanii is not less than 1×10⁸ CFU/mL.

5. The method according to claim 4, wherein the liquid medium is an inorganic salt liquid medium.

6. The method according to claim 5, wherein the inorganic salt liquid medium comprises the following components: KH₂PO₄ 0.7 g/L, K₂HPO₄·3H₂O 0.5 g/L, NH₄Cl 2 g/L, MgSO₄·7H₂O 0.6 g/L, NaCl 0.005 g/L, FeSO₄·7H₂O 0.001 g/L, ZnSO₄·7H₂O 0.002 g/L and MnSO₄·H₂O 0.001 g/L.

7. A product applicable to degrading polyethylene terephthalate, wherein the product contains S. pavanii, and the S. pavanii has been deposited in China Center for Type Culture Collection on Jun. 3, 2019 with the deposit number of CCTCC NO: M 2019415.


SEQUENCE LISTING (1 sequence)

SEQ ID NO: 1 - DNA, 1416 bp, Stenotrophomonas pavanii
  source 1..1416: mol_type = other DNA
ggcagcgccctcccgaaggttaagctacctgcttctggtgcaacaaactcccatggtgtgacgggcggtgtgtacaaggcccgggaacgtattcaccgcagcaatgctgatctgcgattactagcgattccgacttcatggagtcgagttgcagactccaatccggactgagatagggtttctgggattggcttaccgtcgccggcttgcagccctctgtccctaccattgtagtacgtgtgtagccctggccgtaagggccatgatgacttgacgtcatccccaccttcctccggtttgtcaccggcggtctccttagagttcccaccattacgtgctggcaactaaggacaagggttgcgctcgttgcgggacttaacccaacatctcacgacacgagctgacgacagccatgcagcacctgtgttcgagttcccgaaggcaccgatccatctctggaaagttctcgacatgtcaaggccaggtaaggttcttcgcgttgcatcgaattaaaccacatactccaccgcttgtgcgggcccccgtcaattcctttgagtttcagtcttgcgaccgtactccccaggcggcgaacttaacgcgttagcttcgatactgcgtgccaaattgcacccaacatccagttcgcatcgtttagggcgtggactaccagggtatctaatcctgtttgctccccacgctttcgtgcctcagtgtcagtgttggtccaggtagctgccttcgccatggatgttcctcctgatctctacgcatttcactgctacaccaggaattccgctaccctctaccacactctagtcgcccagtatccactgcagttcccaggttgagcccagggctttcacaacggacttaaacgaccacctacgcacgctttacgcccagtaattccgagtaacgcttgcacccttcgtattaccgcggctgctggcacgaagttagccggtgcttattctttgggtaccgtcatcccaaccgggtattagccagctggatttctttcccaacaaaagggctttacaacccgaaggccttcttcacccacgcggtatggctggatcaggcttgcgcccattgtccaatattccccactgctgcctcccgtaggagtctggaccgtgtctcagttccagtgtggctgatcatcctctcagaccagctacggatcgtcgccttggtgggcctttaccccgccaactagctaatccgacatcggctcattcaatcgcgcaaggtccgaagatcccctgctttcacccgtaggtcgtatgcggtattagcgtaagtttccctacgttatcccccacgacagagtagattccgatgtattcctcacccgtccgccactcgccacccagagagcaagctctcctgtgctgccgttcgactgcatgt